(19) United States
(12) Patent Application Publication
(10) Pub. No.: US 2009/0208585 A1
(43) Pub. Date: Aug. 20, 2009

(54) STABILISATION OF BIOLOGICAL MATERIALS

(75) Inventors: Bruce Roser, Cambridgeshire (GB); David Moss, Hertfordshire (GB)

Correspondence Address: KOLISCH HARTWELL, P.C., 200 PACIFIC BUILDING, 520 SW YAMHILL STREET, PORTLAND, OR, 97204, US

(73) Assignee: CAMBRIDGE BIOSTABILITY LIMITED, Cambridge (GB)

(21) Appl. No.: 12/065,493

(22) PCT Filed: Aug. 31, 2006
(86) PCT No.: PCT/GB2006/050266

§ 371 Date: Jan. 23, 2009

(30) Foreign Application Priority Data
Aug. 31, 2005 (GB) GB0517688.8

(51) Int. Cl. A61K 39/29; A61K 9/14

(52) U.S. Cl. 424/499; 424/227.1; 424/489

(57) ABSTRACT

Biological materials such as vaccines can be stabilised in certain glassy materials, soluble in water. It has been proposed to form these glassy materials as a powder suspended in a non-aqueous liquid for injection into a patient. There is a problem in maintaining the suspension because the particles tend to sink to the bottom. The problem is solved by adding a blowing agent into a solution for which the glass is formed. The blowing agent decomposes as the solution evaporates thereby forming cavities in the resulting glass structure, reducing its density to match that of the liquid in which it is to be suspended. Other uses for the invention are in compositions intended for inhalation and for rapid dissolution in aqueous solutions immediately before use.

It is known that certain materials, particularly certain sugars, are capable of forming a glass ie a non-crystalline solid which will stabilise biological material carried by it either in suspension or solid solution. Examples of such biological materials are vaccines and insulin. Sometimes the biological material needs to be presented in liquid form e.g. for injection into a patient. For this purpose it has been proposed to form the glassy material as particles suspended in an inert, non-toxic liquid.

A major problem associated with the above proposal has been the selection of a suitable liquid. Traditional liquid carriers used for injection of medications, are oils which are less dense than the sugar glasses usually used for stabilisation; causing the latter to sink to the bottom of the liquid. This sediment can become very compact with time. Although the container could be re-agitated to re-form the suspension before administration this agitation may need to be very vigorous and prolonged to be effective. There is an unacceptable risk that such agitation might not be performed properly, thus compromising the effectiveness of the medication.

More recently it has been proposed to use perfluorocarbons as the liquid carrier. Perfluorocarbons have a high density, allowing one to match the density of the particles to that of the liquid by adding a more dense material such as calcium phosphate into the glass. However, although this technique has been shown to work well and is suitable for small-scale or emergency purposes, the use of perfluorocarbons in large quantities needs to be undertaken with caution because it could have an undesired effect on an upper part of the earth's atmosphere. These materials are extremely stable even to high fluxes of ultraviolet light and persist in the stratosphere for millennia where they can constitute a global warming hazard.

According to this invention there is provided a composition comprising a body including a biological material and a glassy material that serves to stabilise the biological material characterised in that the body contains at least one gaseous void.

Preferably there are a large number of such “bodies” each being one particle of a powder. With proper control over the manufacturing process, it is possible to reduce the density of these particles to that of a low-density liquid, especially oils, used as liquid carriers. In this way, it is believed that a stable suspension can be obtained using traditional liquids that have been proven to be entirely safe.

It is possible for each particle to have just a single void so that it takes a form somewhat similar to an inflated ball, or for it to be formed as a foam or honeycomb-like structure containing many voids. Alternatively a structure between these two extremes is possible where just a few voids are included in a single particle.

The glassy particles can be formed using a solution of the glass-forming material mixed with the biological material. This mixture is then heated in a dry environment so as to evaporate the solvent (usually water) under conditions so that it solidifies as a non-crystalline solid i.e. a glass. If, during this process, the temperature of the mixture is known at which it is in a transitional state between liquid and solid, an additive can be chosen that forms a gas at this temperature whilst glassification is occurring so that the gas becomes trapped in the glass to form the gaseous voids of the invention.

According to a second aspect of the invention there is provided a method of making a glassy product characterised by the steps of:
i) mixing a first liquid material capable of forming a glass with a second material that can be caused to form a gas; and
ii) causing the first material to form a glass at the same time as the second material forms the gas;
   whereby a glassy structure is formed comprising a glass containing the gas.

There is a very wide variety of glassy materials that can be used. Suitable materials include sugars such as raffinose and trehalose, palatinit (a mixture of glucopyranosyl sorbitol and glucopyranosyl mannitol), glucopyranosyl sorbitol, glucopyranosyl mannitol, lactitol, and monosaccharide alcohols.

A problem associated with the use of sugar glasses to stabilise biological materials is that it is necessary to eliminate almost all of the water from the glass to achieve the desired stabilisation effect. Achieving this can require conditions (e.g. a high temperature) which are not compatible with those required to achieve the glassy state and to avoid damaging the active biological material. We have now discovered that this problem can be solved by using glutamic acid or a salt thereof such as monosodium glutamate (hereinafter referred to as “MSG”) as a component in the glass forming material, instead of sugars.

MSG has previously been recognised as having stabilising properties when mixed with other glass-forming substances as described for example in patent specification U.S. Pat. No. 6,872,357 and in the paper “Recoveries of bacteria after drying in Glutamate and other substances” by D. I. Annear, published in the Aust. J. exp. Biol. Med. Sci (1964), 42. pp. 717-722. However, we have now discovered that MSG can have over 3% of residual moisture and up to about 5% yet still be substantially un-softened by it and retain its stabilising properties up to about 70° C. Furthermore, whilst entering the glassy state, we have found that MSG has a pronounced transitional state where it is in the form of a viscous syrup-like semi-solid. This makes it particularly effective for trapping bubbles of gas as they are formed

The discovery that MSG and similar compounds can be used as a stabiliser even with these relatively high concentrations of water is considered to be independently inventive and thus, according to a further aspect of this invention there is provided a composition in which a biological material is stabilised by a glassy substance comprising glutamic acid or a salt thereof characterised in that the glassy substance contains over 3% by weight of water, a range of between 4% and 5% inclusive being preferred.

Where glutamic acid or a salt such as MSG is used it has been found that the active material (particularly if it includes a microparticulate adjuvant) can cause crystallisation to occur. This difficulty can be overcome by including a crystallisation inhibitor such as aspartic acid or a salt thereof. This crystallisation inhibitor is preferably itself a glass former such as monosodium aspartate (MSA) and the two components are preferably present in similar molar ratios (between 4:6 and 6:4) to give optimum inhibition of crystallisation.

During the process of drying a solution of glass-forming material by heat, especially if the drying is taking place in droplets of the material, the temperature of the droplets is kept from rising by a rapid rate of evaporation from the surface of the droplet with its associated evaporative cooling. As the solution becomes more viscous the mobility of water molecules towards the surface of the droplet is slowed by the increasing viscosity and the temperature of the droplet rises as evaporative cooling reduces. Thus the temperature of the drying syrupy droplet rises rapidly as it begins to solidify as a glass.

The gas is preferably introduced into the glass by mixing the glass-forming material (and the biological material) with a chemical that decomposes under the appropriate conditions to form the gas; but there is also the possibility of using a material which becomes gaseous without undergoing any chemical change. In either case, the additive is preferably chosen so that the gas is released when the temperature has risen to a level at which the glass-forming material is in a viscous transitional state between liquid and solid. Ammonium bicarbonate has been identified as a suitable additive because this decomposes into ammonia gas, carbon dioxide and water vapour at about the same temperature as the increase in viscosity i.e. at about 60° C.

The use of MSG is thus particularly appropriate because the water by-product does not adversely affect its stabilising properties. It may be possible; however, to use other glass forming materials such as sugars including raffinose and trehalose. The use of calcium phosphate is of particular interest because of its physical strength. It is conjectured that a solid honeycomb-like component produced in this way could be moulded so as to form a structural part, especially if formed with a continuous outer surface. It would have great strength and light weight similar to the characteristics of animal bone. Possibly such a substance could be used for bone repair or replacement purposes.

The above description assumes that it is necessary to select an additive that has inherent properties causing it to release the gas at the time when the glass-forming substance is semi-solid i.e. sufficiently viscous to ensure that the gas is trapped. However there is an alternative possibility of using an external influence to trigger the release of gas exactly at the time required. This could be done e.g. by exposing the material to radiation or other stimulation to which it responds by undergoing the physical or chemical change required to cause the release of gas.

Examples of how the invention has been implemented will now be described by way of example with reference to the accompanying illustrations in which:

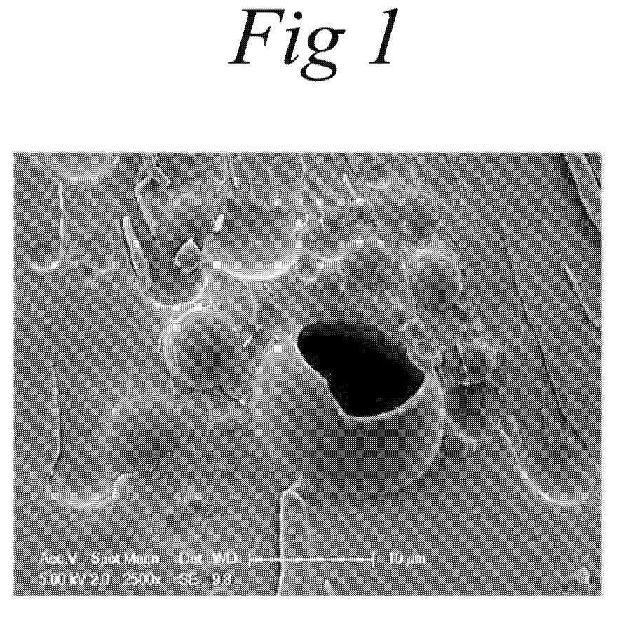

FIG. 1 is a picture produced by a scanning electron microscope, of glassy particles of MSG produced by a method in accordance with the invention;

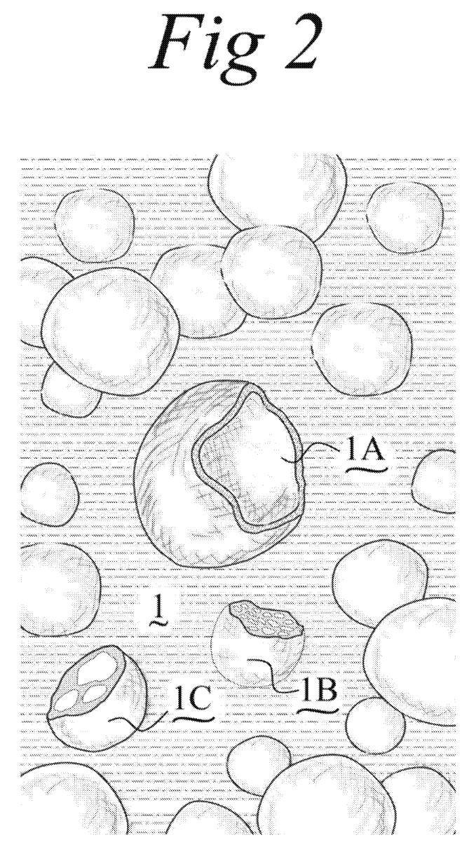

FIG. 2 is a very schematic drawing showing the particles of FIG. 1 suspended in a non-toxic non-aqueous biocompatible liquid to form a stable suspension suitable for injection through a hypodermic syringe; and

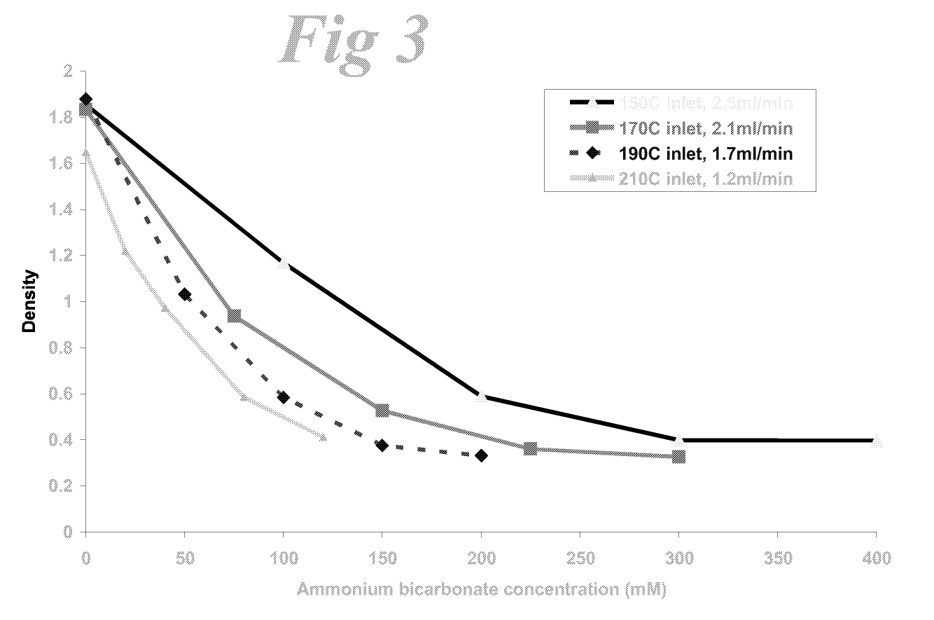

FIG. 3 shows the results of experiments demonstrating how density of the particles varies with varying ammonium bicarbonate concentrations for different spray drier inlet temperatures and flow rates.

The first step is to make an aqueous solution containing a glass forming agent and a gas forming agent (hereinafter referred to as a “blowing agent”). In this example the glass forming agent is MSG and the blowing agent is ammonium bicarbonate. The MSG is at a concentration of 200 mg/ml and the ammonium bicarbonate at a concentration of 17.4 mg/ml. A biological material is then added to the solution. This does not significantly effect the concentrations referred to above.

A Buchi B290 mini spray dryer is set so that gas enters its drying chamber at an inlet gas temperature of 150° C. and leaves at approximately 95° C. The drying gas flow rate is set at a nominal value of 600 litres/hour into the drying chamber.

The aqueous solution is introduced into the drying chamber as a fine spray through a 0.7 mm nozzle. During a period of between 1 and 1.5 seconds this dries to form a powder which is then collected. The powder is separated from the drying gas in a cyclone attached to the exhaust of the drying chamber and falls by gravity into a bottle attached to the bottom of the cyclone. During this period the temperature of the particles rises from room temperature (about 21° C.) to a theoretical maximum of 95 to 120° C. (the outlet temperature). In actual practice the temperature of the dry glass particles does not dwell for any prolonged period at the outlet temperature since they are separated from the hot exhaust gases in the cyclone within a few seconds and collect together in the cooler collection bottle.

As the particles pass though the drying chamber, their temperature initially rises relatively slowly because heat absorbed by the particles is used in the evaporation of the solvent (water). The particles then form into a glass by solvent evaporation, passing through an intermediary stage where they are neither liquid nor glass. In the conditions of the process as described above the particles are at about 60° when they enter this intermediary stage. Continued heating drives off much of the remaining water so that the composition continues to harden through solvent evaporation and soon forms a glass.

Ammonium bicarbonate decomposes when exposed to heat, beginning at 36° C. and fully decomposing at 60° C. The products of decomposition are 21.5% ammonia, 55.7% carbon dioxide and 22.8% water vapour (data from the Merck Index). Thus, between approximately 60° C. and 70° C., just as the particles are in their intermediary state between liquid and glass, the ammonium bicarbonate is decomposing rapidly into ammonia, carbon dioxide and water vapour. At this time, the MSG is soft and pliable. It is semi-solid like thick syrup. Gas bubbles are therefore trapped within the MSG droplets as they enter a glassy state.

Typical moisture content of the spray-dried final product made by the process as described above is around 4% by weight, measured by the Karl Fischer coulometry method. The particles have a mean density of 0.94 (compared to MSG alone which has a density of 1.46). Powder densities can be measured by pycnometry using helium displacement.

Finally, the dried powder, containing the biological material stabilized by the effect of the glassy MSG, is suspended in a non-aqueous mixture of medium chain triglycerides comprised of approximately 60% caprylic (C8, octanoyl) acid and 40% capric (C10, decanoyl) acid. This mixture is sold under the trade name of “Crodamol GTCC”) and is an anhydrous, non-toxic biocompatible liquid having a density approximately equal to the spray dried spheres. The density of the Crodamol GTCC is sufficiently close to that of each of the spheres, to have the effect that once suspended in the liquid, the spheres are all retained in suspension at normal temperatures. The suspension is so physically and biologically stable that it can be used in pre-filled injectors and stored and transported without refrigeration. Because of the chemical stabilising effect of the MSG, the active ingredient does not deteriorate and, because of the effect on density of the gas voids in the particles, the latter remain indefinitely in suspension. It will be appreciated that, when the suspension has been injected into an human or animal body, the glass particles, being soluble in water, will dissolve in body fluid, thereby releasing the active ingredient.

FIG. 1 shows a picture, produced using a scanning electron microscope, of spray dried particles manufactured according to the method described above. To inspect the interior structure of the spheres, they were mixed with a carrier liquid which was then frozen and broken in accordance with a standard “freeze-fracture” practice used in microscopy. The broken surface of the frozen carrier forms the background of the picture. It can be seen that the particles are approximately spherical and vary in diameter from about 2 μm to about 15 μm sufficiently small that the suspended particles will pass through the needle of a hypodermic syringe. The size of the particles noted above should be compared with an average particle size of about 3-5 μm that are produced using a similar method but without the ammonium bicarbonate.

In FIG. 1, one of the largest spheres is broken, revealing that it is entirely hollow and has a wall thickness of about 1 μm. It is possible that the smaller spheres may have a similar structure or a more complex structure as illustrated in FIG. 2.

Referring to FIG. 2, the Crodamol GTCC liquid is indicated at 1 and has, suspended in it, glass particles 2 having a mean density equal to that of the liquid. Particle 2A, shown broken away to reveal its interior, is structured like that shown in FIG. 1. Particle 2B, also shown broken away, contains a large number of gaseous voids, forming a structure like a sponge or honeycomb; whilst particle 2C has a structure in-between the extremes of 2A and 2B, having just a few voids. The densities of these different structures will be slightly different but they are all within a range that allows thermodynamic processes at normal temperatures to keep the particles in permanent suspension.

In an alternative process according to the invention, instead of employing pure MSG, a mixture of an equal (0.5 M) molar solution of MSG and MSA is formed by the addition of 0.935 g of MSG and 0.865 g MSA (total 1.8 g) to 10 mL of distilled water. Suitable materials include Monosodium L-glutamate monohydrate and monosodium L-aspartate monohydrate from Ajinomoto.

The resulting solution is added to a vaccine material which comprises 2 mg of a hepatitis B antigen with 160 mg of aluminium hydroxide adjuvant so as to give a ratio of total stabiliser (i.e. in this case MSA and MSG) to adjuvant of 10.7:1 (1.8 g:0.16 g). An amount of ammonium bicarbonate within the range shown in FIG. 3 is added.

A Buchi B290 mini spray dryer is then used as previously described to form a low density powder.

As, in either of the described processes, the glass is formed from water soluble glass formers, the resulting powder can be rehydrated when needed with the required amount of sterile water.

It will be appreciated that the processes described above are just examples of how the invention may be used. The density of the particles can be precisely controlled, by the addition of a selected quantity of ammonium bicarbonate, so as to match their density with that of any alternative liquid in which they are to be suspended. Experiments have shown that it is possible to incorporate up to 1.0 M (79 mg/ml) ammonium bicarbonate, that latter concentration producing dried spheres with a mean diameter of approximately 20 μm and a density of 0.64. Other studies, the results of which are shown on FIG. 3, have shown that the effect of varying the ammonium bicarbonate concentration, varying the flow rate of the liquid entering the spray drier and varying the inlet gas temperate of the spray drier is to closely control the density of the microspheres produced.

Although ammonium bicarbonate has been described as the agent used to create the voids in the particles, it is observed that many other materials could be used to achieve a similar effect, the essential requirement being only that the gas should be released at the same time that the glass-forming substance is in a viscous semi-solid or semi-liquid form such that bubbles or voids of gas will become trapped in it. Whilst the application of heat is a convenient mechanism by which the release of gas can be triggered from the blowing agent, there may be alternative mechanisms, such as the use of irradiation microwaves or ultrasound, for achieving the same effect irrespective of temperature. Also, whilst the described example utilises a chemical reaction to generate the gas, this could alternatively be achieved by a mere change of state of a suitable additive, from a solid or liquid into a gas.

Although particular sugars and amino acids have been mentioned as the glass formers, there are many alternative known materials that are capable of providing the required effect of biological stabilisation by formation of a glassy medium.

It is also observed that although, in the particular example described, the gas is released whilst glass is being formed by solvent evaporation, a similar effect may be achievable by using glasses whose transition temperature is matched to the temperature of the gas forming substance (the blowing agent). In such a variation, glass, mixed with the blowing agent would be heated so that it softened, at which point the gas would be released to form the required voids. Subsequent cooling would complete the process.

The techniques described above provide a promising way of adjusting the density of particles so as to match the density of a liquid in which they are subsequently suspended. However, it has been observed that particles produced in accordance with the invention can be of such a low density that they are easily suspended in a gaseous medium and that the invention is therefore of potential application in medications intended for inhalation. The particles, because of their large surface area (including areas exposed to the internal voids), have also been noted as having the ability to dissolve rapidly in aqueous liquids and may therefore be of value in situations where it is desired to store stabilised biological materials in solid stable form and to dissolve them shortly before use.

1. A composition comprising a body including a biological material and a glassy material that serves to stabilise the biological material characterised in that the body contains at least one gaseous void.

2. A composition according to claim 1 comprising a plurality of such bodies, in the form of particles.

3. A composition according to claim 2 characterised in that the particles are suspended in a liquid.

4. A composition according to claim 3 characterised in that the liquid is less dense than solid parts of the particles and further characterised in that the voids cause the densities of the particles to be closer to the density of the liquid.

5. A composition according to claim 4 characterised in that the densities of the particles and of the liquid are sufficiently similar that, at normal temperatures, the particles remain permanently in suspension.

6. A composition according to any preceding Claim characterised in that there are a plurality of voids whereby the glassy material defines a foam or honeycomb structure.

7. A composition according to any one of claims 2 to 5 characterised in that the glassy material is in the form of hollow particles having a solid outer shell and a hollow interior

8. A composition according to any preceding Claim characterised in that the glassy material includes glutamic acid or a salt thereof.

9. A composition according to any one of claims 1 to 7 characterised in that the glassy material includes a sugar.

10. A composition according to any one of claims 1 to 7 characterised in that the glassy material includes glutamic acid or a salt thereof and a crystallisation inhibitor.

11. A composition according to any one of claims 1 to 7 characterised in that the crystallisation inhibitor is itself a glass former.

12. A composition according to claim 11 characterised in that the crystallisation inhibitor includes aspartic acid or a salt thereof.

13. A composition according to any preceding Claim in which the glassy material includes calcium phosphate.

14. A composition according to any preceding Claim characterised in that the void or voids contain gaseous products of the decomposition of ammonium bicarbonate.

15. A method of making a glassy product characterised by the steps of:
i) mixing a first, liquid, material capable of forming a glass with a second material that forms a gas when heated; and
ii) causing the first material to form a glass at the same time as the second material forms the gas; whereby a glassy structure is formed comprising a glass containing the gas.

16. A method according to claim 15 characterised in that the glass includes monosodium glutamate.

17. A method according to claim 15 or 16 characterised in that the glass includes calcium phosphate.

18. A method according to claim 15, 16 or 17 characterised in that the second material is such that it decomposed at a time when the first material is in a transition state between liquid and glassy solid.

19. A method according to claim 15, 16, 17 or 18 characterised in that the second material includes ammonium bicarbonate.

20. A method according to any one of claims 15 to 19 characterised by the step of forming the glassy structure into particles containing the gas.

21. A method according to any one of claims 15 to 20 characterised by the step of forming the glassy structure into a structural body.

22. A method according to any one of claims 15 to 21 characterised in that the particles are formed in a spray drier.

23. A method according to any one of claims 15 to 22 characterised by the further step of suspending the particles in a non-toxic liquid.

24. A composition in which a biological material is stabilised by a glassy substance comprising glutamic acid or a salt thereof characterised in that the glassy substance contains over about 3% by weight of water.

25. A composition according to claim 24 characterised in that the glassy substance comprises MSG.

26. A composition according to claim 24 or 25 characterised in that the glassy substance contains over about 5% by weight of water.

27. A composition according to claim 26 characterised in that the weight of the water is over 3% of the weight of the glassy substance.

28. A composition according to claim 27 characterised in that the weight of the water is over 5% of the weight of the MSG.
